(19) United States
(12) Patent Application Publication
(10) Pub. No.: US 2024/0131230 A1
(43) Pub. Date: Apr. 25, 2024

(54) ACELLULAR SKIN SUBSTITUTE FOR BREAST RECONSTRUCTION AND PREPARATION METHOD THEREFOR

(71) Applicant: L&C BIO Co., Ltd., Gyeonggi-do (KR)

(72) Inventors: Sol LEE, Seoul (KR); Ki Min KIM, Yongin-si Gyeonggi-do (KR); Sang Yeol KIM, Gyeonggi-do (KR); Kwang Hoon SHIN, Gyeonggi-do (KR); Hyun Seung LEE, Gyeonggi-do (KR); Hyung Gu KIM, Seoul (KR); Whan Chul LEE, Seoul (KR)

(21) Appl. No.: 18/277,243

(22) PCT Filed: Feb. 15, 2021
(86) PCT No.: PCT/KR2021/001866

§ 371 Date: Aug. 14, 2023

(51) Int. Cl. A61L 27/60; A61F 2/12; A61L 27/36

(52) U.S. Cl. CPC A61L 27/60; A61F 2/12; A61L 27/362; A61L 27/3687; A61L 27/3691; A61F 2220/0075; A61F 2230/0006; A61F 2230/0023; A61F 2240/001; A61L 2430/04; A61L 2430/40

(57) ABSTRACT

The present invention relates to an acellular skin substitute for breast reconstruction and a preparation method therefor.

In the present invention, an acellular skin substitute may be prepared through the removal of epidermal layers and an acellularization process, in which the use of a proteolytic enzyme and a surfactant is excluded. In addition, since the acellular skin substitute is cut into suitable sizes and shapes to cover a breast reconstruction implant through design software and then cut out with a CNC machine, the time for breast reconstruction surgery may be reduced, and since no dead space is formed after suturing, the incidence of seromas may be reduced. Accordingly, the side effects of surgery may be lessened.

### TECHNICAL FIELD

The present invention relates to an acellular skin substitute for breast reconstruction and a method of preparing the same.

### BACKGROUND ART

An acellular dermal matrix (ADM) is a dermal matrix obtained from human or animal skin through acellularization technology, and refers to an extracellular matrix (ECM)-type bio-derived skin substitute consisting of pure collagen and elastin. Unlike organs for transplantation, such bio-derived skin substitutes are known to have relatively low immune rejection. Although there is a difference in the degree of immune rejection when transplanting a human-derived skin substitute and an animal-derived skin substitute, it is generally reported that a human-derived skin substitute is superior to an animal-derived skin substitute in terms of biological performance and safety. However, since a human-derived skin substitute has to be obtained by posthumous donation of skin tissue from a donor, there may be limitations in terms of the supply and demand of raw materials.

On the other hand, in most medical fields requiring surgery, the demand for human tissue derived from humans is increasing day by day. Particularly, it has been reported that the demand for skin is rising rapidly, and the dependence on imports of raw materials and finished human tissue products has reached 91.5%. According to data from the Korea Health Insurance Review and Assessment Service, the size of the ADM domestic market in 2016 (based on insurance benefits, excluding non-coverage benefits) is estimated at approximately 50 to 100 billion won for a single item. In accordance with this trend, the development of medical appliances such as artificial skin or wound dressings is actively being conducted, but a skin substitute that can surpass the biological performance of actual human-derived ADM has not yet been reported. The global market size of these medical appliances, which are skin substitutes, is estimated at 1.8 billion dollars in 2016.

One of the reasons for the expansion of the human-derived ADM market at home and abroad is the increase in breast cancer patients, which is ranked first in the world. After a total mastectomy for the treatment of breast cancer, breast reconstruction is implemented. In addition, upon breast reconstruction, to maintain the volume and shape of breasts, implants such as silicone are used, and a human-derived ADM or animal-derived ADM is used to prevent side effects such as adhesion and capsular contracture. More than 6,000 breast reconstruction surgeries are conducted annually in Korea, and an implant is surrounded by an ADM and then inserted into a breast reconstruction area. As described above, due to the attitude of preferring human-derived ADMs over animal-derived ADMs in the medical field, the demand for human-derived ADMs continues to increase.

A two-step process of obtaining an ADM is known in general. The first step is a step for removing the epidermis from prepared skin tissue, in which the epidermis and the dermis of the skin are separated by changing the ionic strength of a proteolytic enzyme or solution.

The second step is a step for removing cells of the dermis after separating the epidermis in the first step. An immune response is mainly caused by membrane proteins present in the cell membrane and may be minimized by removing the cells. The above step uses a method of selectively removing only cells without tissue damage by using the difference in physicochemical properties between cells and the extracellular matrix. The main component of the cell membrane is phospholipids, and cells may be removed without tissue damage using various surfactants or enzymes.

The processed ADM is cut by medical staff to fit the size of an implant to be inserted into a patient in an operating room for breast reconstruction. The medical staff wraps the implant to be applied to the patient with an ADM, and in the wrapping process, the overlapping parts of the dermis and excessive margins are removed. Since the designing and cutting of an ADM to fit the implant by the medical staff takes a lot of time, the operation time becomes longer and many ADMs are discarded without being used for a patient. Accordingly, the operation cost and the consumption of limited resources increase.

Therefore, the present invention is intended to provide a method of removing the epidermis and cells from donated skin at one time, not sequentially, by base treatment without using enzymes or surfactants. In addition, to eliminate the time to design and cut an ADM to fit a patient's implant at the site of a breast reconstruction surgery, the present invention is intended to provide a method of preparing an acellular skin substitute in an appropriate design and size to reduce the surgery time and save the acellular skin substitute that has been donated and processed.

In addition, the present invention is intended to provide a method of minimizing surgical side effects by reducing seromas occurring due to a dead space since an acellular skin substitute is tightly adhered and covers a breast reconstruction implant that will be inserted into a patient.

### DISCLOSURE

### Technical Problem

The present invention provides a method of preparing an acellular skin substitute optimized in shape and size for breast reconstruction surgery.

More specifically, the present invention is intended to obtain an acellular skin substitute by the separation of the epidermis and an acellularization process without the use of a surfactant and an enzyme, and to provide an acellular skin substitute in a design and shape that can wrap a breast reconstruction implant without designing and cutting the substitute in breast reconstruction surgery.

### Technical Solution

The present invention provides an acellular skin substitute, which includes: a circular base part; and

three or more protrusions which protrude outward from the circumference of the base part, and are provided at different positions along the circumferential direction of the base part,

wherein a plurality of slits are formed radially along the circumferential direction of the base part based on the center of the base part in the base part and at least one of the protrusions,

each slit has a start point and an end point, and

an angle between a first imaginary line extending from the center of the base part to the center of the slit and a second imaginary line connecting the start and end points of the slit is 60 to 120°.

In addition, the present invention provides a method of preparing an acellular skin substitute, which includes: placing an acellular dermal matrix (ADM) on a cooling plate maintained at−30 to 0° C.;
adding water onto the cooling plate to reach 20 to 150% of the thickness of the ADM and freezing the ADM; and
cutting the frozen ADM using a cutting device.

In addition, the present invention provides a method of preparing an acellular skin substitute, which includes: preparing an ADM by removing the epidermis and cells from skin tissue;
placing the ADM on a cooling plate maintained at−30 to 0° C.;
adding water onto the cooling plate to reach 20 to 150% of the thickness of the ADM and freezing the ADM;
setting the shape and size of an acellular skin substitute that is able to wrap a breast reconstruction implant using a design program; and
cutting the frozen ADM into the shape and size set by the design program using a cutting device,
wherein the shape of the ADM set by the design program includes a circular base part; and
three or more protrusions which protrude outward from the circumference of the base part, and are provided at different positions along the circumferential direction of the base part,
a plurality of slits are formed radially along the circumferential direction of the base part based on the center of the base part in the base part and at least one of the protrusions,
each slit has a start point and an end point, and
an angle between a first imaginary line extending from the center of the base part to the center of the slit and a second imaginary line connecting the start and end points of the slit is 60 to 120°.

### Advantageous Effects

In the present invention, an acellular skin substitute can be prepared by the removal of the epidermis and an acellularization process without using a protease and a surfactant.

The acellular skin substitute is used by being designed to have an appropriate size and shape for wrapping a breast reconstruction implant by a design program and being cut with a CNC machine, so the time for breast reconstruction surgery can be reduced. Since no dead space is formed after suturing, the incidence of seromas can be reduced. Accordingly, the side effects of surgery can be lessened.

In addition, the acellular skin substitute according to the present invention serves as a scaffold after implantation, and can serve as a physiological space in which implanted cells can migrate and grow. In addition, the acellular skin substitute becomes the tissue of a patient who has received the implantation over time due to angiogenesis, so it can be used as a skin substitute for the repair and plastic surgery of damaged tissue.

### DESCRIPTION OF DRAWINGS

FIG. 1 shows the shape of an acellular skin substitute according to one embodiment of the present invention.

FIG. 2 shows the results obtained by observing the epidermis and cell nuclei after immunohistochemical staining and DAPI staining, respectively, using an optical microscope and a fluorescence microscope, to confirm the removal of the epidermis and cells from skin tissue after treatment with a decellularization solution.

FIG. 3 shows the SEM results to confirm the deformation or damage of collagen fibers after treatment of a decellularization solution.

FIG. 4 shows the result of quantifying DNA, which is extracted from skin tissue, after treatment with a decellularization solution.

FIG. 5 shows the technical illustration of breast reconstruction by wrapping a breast reconstruction implant with an acellular skin substitute having a design and a shape, which have been developed by the present invention.

FIG. 6 is a CAD drawing that shows the shape of an acellular skin substitute capable of wrapping and suturing a breast reconstruction implant.

FIG. 7 is a photograph showing a cooling plate manufactured to maintain an acellular skin substitute at −20° C.

FIG. 8 is a photograph showing the result of freezing an acellular skin substitute after pouring water on a cooling plate to reach an ADM thickness to fix an acellular skin substitute placed on the cooling plate so it does not move during the operation of a CNC machine.

FIG. 9 is a photograph showing the process of cutting an acellular skin substitute according to the CAD drawing to properly wrap a breast reconstruction implant.

FIG. 10 is a photograph of an acellular skin substitute cut with a CNC machine according to the CAD drawing. The cut acellular skin substitute may wrap a breast reconstruction implant and may be sutured.

FIG. 11 is a photograph showing the upper side of a breast reconstruction implant wrapped with an acellular skin substitute that has been designed and cut according to the present invention.

FIG. 12 is a photograph showing the back side of an acellular skin substitute designed and cut according to the present invention, which wraps a breast reconstruction implant.

FIG. 13 is a set of photographs of cutting an acellular skin substitute subjected to a freezing process (b) and an acellular skin substitute not subjected to a freezing process (a) using a CNC machine.

### MODES OF THE INVENTION

The present invention relates to an acellular skin substitute, which includes: a circular base part; and
three or more protrusions that protrude outward from the circumference of the base part and are provided at different positions along the circumferential direction of the base part,
wherein a plurality of slits are formed radially along the circumferential direction of the base part based on the center of the base part in the base part and at least one of the protrusions,
the slit has a start point and an end point, and
an angle between a first imaginary line extending from the center of the base part to the center of the slit and a second imaginary line connecting the start and end points of the slit is 60 to 120°.

Hereinafter, the acellular skin substitute according to the present invention will be described in further detail.

In the present invention, the acellular skin substitute may be in the form of a sheet and may be used to wrap a breast reconstruction implant.

In the present invention, the acellular skin substitute is a dermal matrix obtained from human or animal skin through decellularization technology and may be expressed as ADM.

In one embodiment, a commercially available ADM product may be used as an acellular skin substitute, or an acellular skin substitute may be prepared by removing the epidermis and cells from skin tissue, and used.

In one embodiment, the acellular skin substitute may be prepared by removing the epidermis and cells from skin tissue.

The removal of the epidermis and cells may be prepared by a general process of removing the epidermis and cells used in the art. In the present invention, the epidermis and cells may be removed using a decellularization solution, and particularly, NaOH.

The removal of the epidermis and cells from the skin tissue will be described in further detail in the method of preparing an acellular skin substitute, which will be described below.

The acellular skin substitute according to the present invention includes a circular base part; and
three or more protrusions which protrude outward from the circumference of the base part, and are provided at different positions along the circumferential direction of the base part.

The base part and the protrusions are composed of one sheet, only to be distinguished to more easily explain the shape of the acellular skin substitute according to the present invention.

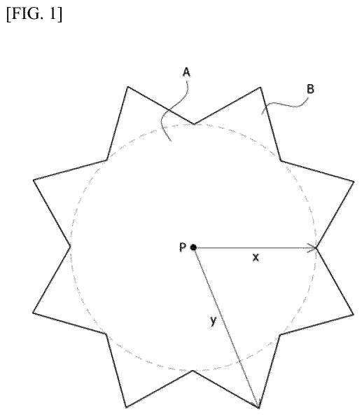

In the present invention, FIG. 1 shows the shape of an acellular skin substitute according to one embodiment of the present invention. In the present invention, the shape of the acellular skin substitute will be described with reference to FIG. 1.

In the present invention, the base part is the A region in FIG. 1, and the base part has a circular shape. The circular shape is not limited to a perfect circle, and may mean any shape without angles, including an elliptical shape.

In one embodiment, the radius (x) of the base part may be 3 to 10 cm. Within the above radius range, the base part is used for human breast reconstruction and may stably wrap the breast reconstruction implant.

In the present invention, the protrusion is the B region of FIG. 1, and there are three or more protrusions that protrude outward from the circumference of the base part, and are provided at different positions along the circumferential direction of the base part. During breast reconstruction surgery, a breast reconstruction implant is wrapped with an acellular skin substitute, and in the wrapping process, the overlapping parts of the dermis and excessive margins are removed, and many ADMs are discarded without being used for a patient. In the present invention, the formation of the protrusions in the acellular skin substitute may minimize the amount of use of the acellular skin substitute, and reduce the time to design and cut an ADM to fit the implant during surgery. According to the present invention, surgery time and costs may be reduced.

In one embodiment, three or more protrusions may be formed, and each protrusion may have the same shape.

In one embodiment, 4 to 12 or 6 to 10 protrusions may be formed, and an even number of protrusions may be formed. When having an even number of protrusions, the acellular skin substitute may have a symmetrical structure.

In one embodiment, one protrusion may form a surface by connecting lines starting from two arbitrary points on the circumference of the base part. Here, one protrusion may not intersect with another protrusion except for the two arbitrary points.

In one embodiment, the protrusion may have a structure in which the area becomes smaller as it extends from the circumference of the base part in the protruding direction. Specifically, the protrusion may have a pointed end (tip) formed toward the protruding direction. The end of this protrusion may be expressed as a vertex. The vertex may be connected to the center of the base part in a straight line.

In one embodiment, the vertices of the three or more protrusions may have the same angle.

In one embodiment, the protrusion may have a triangular shape where two straight lines originating from two arbitrary points on the circumference of the base part meet.

In one embodiment, the length (y) between the center point of the base part and the end of the protrusion may be 3 to 15 cm. In addition, the ratio of the radius (x) of the base part and the length (y) between the center point of the base part and the end of the protrusion, i.e., x:y, may be 1:1 to 1:2 or 1:1 to 1:1.5. Within the above range, the upper surface of a breast reconstruction implant may be covered, and it is advantageous for suturing the end of the protrusion on the backside. Here, in the breast reconstruction implant, the side where the nipple is located may be referred to as the upper side, and the side located toward a patient's ribs may be referred to as the back side.

In one embodiment, the thicknesses of the base part and the protrusions may be 0.5 to 5 mm. Within the above range, the breast reconstruction implant may be stably wrapped, the protrusions of the skin substitute are possibly sutured together, and the mechanical properties may be maintained.

In addition, in the acellular skin substitute of the present invention, in the base part and at least one of the protrusions, a plurality of slits may be formed radially along the circumference direction of the base part based on the center point of the base part.

In the present invention, the slit is a narrow gap made by two facing blades, and refers to an incision or penetration. Due to the slit, the area of an acellular skin substitute may increase by 20 to 100%. Specifically, when the breast reconstruction implant is wrapped with the acellular skin substitute, since there is no implant lifting phenomenon, caused by the slit, a dead space is not generated. Accordingly, it is possible to prevent fluid retention, and inhibit inflammatory responses and infections. That is, a spherical implant may be most appropriately wrapped through the slits. In addition, the slits may provide flexibility to obtain a stretching effect, and due to the flexibility, pain and discomfort may be reduced even after implantation. Particularly, since the microscopic spaces of the slits provide an advantageous environment for the proliferation of fibroblasts and the generation of new blood vessels during the implantation process, they may enable rapid recovery after surgery, reduced side effects, reduced foreign body sensation, and natural implantation.

In one embodiment, the formation position of the slit may be optionally designed according to the purpose of use, and specifically, the formation position of the slit may be designed so that the breast reconstruction implant can be wrapped stably without any space.

In one embodiment, 100 to 400 slits may be formed in the acellular skin substitute.

In one embodiment, each slit has a start point and an end point, and the angle between a first imaginary line extending from the center point of the base part to the center point of the slit and a second imaginary line connecting the start point and the end point of the slit may be 60 to 1200 or 80 to 100°.

In one embodiment, two arbitrary points on the circumference of the base part forming one protrusion and the center point of the base part form an imaginary triangle, and in the imaginary triangle, the first imaginary line extending from the center point of the base part to the center point of the slit and the second imaginary line connecting the start point and the end point of the slit may be perpendicular to each other. For example, as many imaginary triangles as the number of protrusions may be set in the base part, and slits having the same shape and size may be formed in the imaginary triangles.

In one embodiment, the shape of the slits is not particularly limited, and one or more shapes selected from the group consisting of a linear shape, a curved shape, and a V shape (comb pattern).

In one embodiment, the length of each slit may be, but not particularly limited, 1 to 20 mm. In addition, the distance between slits may be 1 to 5 mm.

In one embodiment, the acellular skin substitute according to the present invention may be expressed as having a star shape.

In one embodiment, when the shape of the acellular skin substitute according to the present invention is expressed differently, the acellular skin substitute may include a circular base part; and
three or more protrusions that protrude outward from the circumference of the base part and are provided at different positions along the circumferential direction of the base part, and
it can be expressed that the center of the base part is set as a center point, two or more imaginary circular lines having different radii are set with the center of the base part as a center point, and a slit is formed in contact with at least a part of the imaginary circular line.

In one embodiment, the imaginary circular line may have a circular gating structure with the center of the base part as a center point. The “grating structure” refers to a pattern with a periodic or quasi-periodic structure, and the circular grating structure may refer to a pattern formed of two or more circular lines based on the center point. Here, two or more imaginary circular lines may be formed to be spaced apart from each other at regular intervals.

In one embodiment, a plurality of slits may be formed on one imaginary line, and the shape and size of the slits may be the same as described above.

In one embodiment, the distance between the centers of the slits contacting the circumference of the imaginary circular line may be 3 to 30 mm, and the distance between the neighboring imaginary circular lines may be 1 to 5 mm. The elongation effect is excellent with the above length of the slit and the above distance between the slits, the possibility of generating a dead space is low, and the skin substitute may not be torn during suturing of the protrusions.

In one embodiment, when the shape of the acellular skin substitute according to the present invention is expressed differently, it can be expressed that the acellular skin substitute has a polygonal shape, and a first interior angle larger than a straight angle and a second interior angle smaller than the straight angle are alternately formed on the circumference of the polygonal shape. Here, the second interior angle smaller than the straight angle indicates the vertex of a protrusion. The length (x) from the center of the polygonal shape to the vertex forming the second interior angle may be 3 to 10 cm, and the length (y) from the center of the polygonal shape to the vertex forming the first interior angle may be 3 to 15 cm.

The acellular skin substitute according to the present invention may be used to wrap a breast reconstruction implant. Therefore, side effects such as adhesion and capsular contracture may be prevented.

In one embodiment, the breast reconstruction implant may be a general implant used in the art.

In one embodiment, the breast reconstruction implant may be wrapped with the acellular skin substitute, and a protrusion may be fixed (sutured) with surgical thread, and then inserted into a breast reconstruction site. Here, suturing may be performed by fixing protrusions of the acellular skin substitute, and specifically, end portions of the protrusions with suture.

Specifically, after covering the upper surface of the implant with the base part of the acellular skin substitute and allowing the ends of the protrusions to gather on the back side, the ends may be fixed with surgical thread.

As the surgical thread, a thread used in the art may be used without limitation, and specifically, an absorbent surgical thread, i.e., polyglycolide suture, may be used.

That is, the present invention may provide a breast reconstruction implant material, which includes a breast reconstruction implant; and an acellular skin substitute according the present invention, which wraps the breast reconstruction implant, wherein the ends of protrusions of the acellular skin substitute are gathered on the back side of the implant and are fixed with surgical thread.

In addition, the present invention relates to a method of preparing an acellular skin substitute, which includes: (A) placing an ADM on a cooling plate maintained at −30 to 0° C.;
(B) adding water onto the cooling plate to reach 20 to 150% of the thickness of the ADM and freezing the ADM; and
(C) cutting the frozen ADM using a cutting device.

An ADM, that is, an acellular skin substitute, is a dermal matrix obtained by acellularization technology from human or animal skin. A commercially available ADM product may be used as such ADM, or an ADM may be used by removing the epidermis and cells from skin tissue.

That is, in the present invention, a process of preparing an ADM by removing the epidermis and cells from skin tissue (hereinafter, removal of the epidermis and cells) may be further performed.

In the present invention, skin tissue may be allogeneic or heterogeneous skin tissue. The allogeneic may mean human, and the heterogenous may mean a mammal such as an animal excluding a human, that is, a pig, a cow, or a horse.

In the present invention, an acellular skin substitute may be prepared according to the preparation method of the present invention using allogeneic or heterogenous skin tissue.

In the present invention, before the removal of the epidermis and cells from skin tissue, a process of pretreating skin tissue (hereinafter, pretreatment) may be further performed.

The pretreatment may include one or more steps among washing skin tissue; removing fascial tissue, adipose tissue and other foreign substances, attached to the dermis, using a scrapper; and removing remaining unnecessary tissue using scissors and tweezers.

In one embodiment, in the pretreatment, fascial tissue, adipose tissue and other foreign substances may be removed from the skin tissue until the dermis can be seen.

In the present invention, in the removal of the epidermis and cells, the epidermis may be isolated and decellularized using a decellularization solution. Decellularization means the removal of cell components excluding the extracellular matrix, for example, a nucleus, a cell membrane, nucleic acids, etc. from skin tissue.

In one embodiment, as a decellularization solution, a basic solution may be used, and specifically, one or more selected from the group consisting of sodium hydroxide, potassium hydroxide, ammonium hydroxide, calcium carbonate, magnesium hydroxide, calcium hydroxide, and ammonia may be used. In the present invention, as a decellularization solution, sodium hydroxide (NaOH) may be used. Conventionally, decellularization was performed using a surfactant and an enzyme. However, when an enzyme is used, it can damage the dermis itself, and when the enzyme remains and is injected into the body, it can damage the original tissue of a patient in severe cases, there is a problem causing an immune response. In addition, when using a surfactant, washing is required to minimize the remaining surfactant, and the remaining surfactant may be the cause of in vivo cell and tissue toxicity. Therefore, in the present invention, the above-described problems may be solved by using a decellularization solution during decellularization, and also has the advantage of no cytotoxicity.

In one embodiment, the concentration of the decellularization solution may be 0.01 to 1 M or 0.05 to 0.5 M. Within this concentration range, it is easy to remove cells.

In addition, in one embodiment, the decellularization may be performed for 30 minutes to 12 hours or 1 to 6 hours. Within this time range, it is easy to remove cells.

In the present invention, step (A) is a process of placing an ADM on a cooling plate maintained at −30 to 0° C.

In one embodiment, the temperature of the cooling plate may be maintained at −30 to 0° C. or −25 to −5° C. by allowing a cooling fluid to flow around the cooling plate or be in contact with the surface of the cooling plate.

In one embodiment, the ADM may be placed on a cooling plate and kept flat without the tissue lifting.

In the present invention, step (B) is a process of freezing the ADM by adding water to reach 20 to 150% of the thickness of the ADM on the cooling plate.

Since an ADM is a biomaterial, which is not hard but soft, it is difficult to precisely cut the ADM with the blade of a cutting device. For this reason, a method of cutting it with a CNC machine after freeze-drying the ADM may be used, but according to this method, since freezing is performed at −60° C. or less, during the freeze-drying process, ADM's unique collagen structure is destroyed, and the tissue becomes loose and is possibly easily degraded in vivo. In addition, tissue stiffness, which may occur in dehydration, may occur. To solve the above problem, in the present invention, a hydrated form of ADM is frozen by a method of placing an ADM on a cooling plate that can be maintained at−30 to 0° C. and adding water. Therefore, the ADM becomes hard and then is cut using a cutting device.

Particularly, in the present invention, since an ADM is frozen using water, ADM may be maintained flat on a cooling plate without using a separate means such as negative pressure, and can also be cut even at−30 to 0° C.

In one embodiment, water may be added to reach 20 to 150% or 50 to 100% of the ADM thickness. Within the above content range, by freezing an ADM, the ADM becomes hard, and the water around the ADM is frozen, so while the ADM is cut with the blade of a cutting device, the ADM does not move, and thus the ADM may be cut to the intended shape and size.

In one embodiment, the freezing of the ADM may be performed for 1 to 6 hours.

In the present invention, step (C) is a process of cutting a frozen ADM using a cutting device.

In one embodiment, the cutting device may be a CNC machine having a round blade with a diameter of 0.1 to 5 mm.

In the present invention, ADM may be cut into a shape and size set by a design program. Such a design program may be a CAD program.

In one embodiment, when the ADM is used to wrap the breast reconstruction implant, the ADM may be designed such that the ADM can be properly wrapped without being lifted or detached from the breast reconstruction implant, and the design may be converted into a CAD program so that it can be run on a CNC machine.

In addition, the present invention relates to a method of preparing an acellular skin substitute for breast reconstruction, which includes: (a) preparing an ADM by removing the epidermis and cells from skin tissue;
(b) placing the ADM on a cooling plate maintained at−30 to 0° C.;
(c) adding water on the cooling plate to reach 20 to 150% of the ADM thickness and freezing the ADM;
(d) setting the shape and size of an acellular skin substitute that is able to wrap a breast reconstruction implant using a design program; and
(e) cutting the frozen ADM into the shape and size set by the design program using a cutting device.

In one embodiment of the present invention, a breast reconstruction implant was wrapped with the acellular skin substitute prepared according to the present invention and fixed with surgical thread. In addition, accordingly, it was confirmed that the acellular skin substitute according to the present invention can be simply used to wrap the breast reconstruction implant.

In the present invention, steps (a), (b), and (c) are the same as (A) and (B), which are the above-described steps of removing the epidermis and cells.

In the present invention, step (d) is a process of setting the shape and size of an acellular skin substitute for breast reconstruction that can wrap a breast reconstruction implant using a design program. Such a design program may be a CAD program.

The ADM according to the present invention is used to wrap the breast reconstruction implant, the ADM may be designed such that the ADM can be properly wrapped without being lifted or detached from the breast reconstruction implant, and the design may be converted into a CAD program so that it can be run on a CNC machine.

In one embodiment, considering that the volume of the breast reconstruction implant is 300 to 600 cc, a design program may be run to prepare the ADM of the present invention with various sizes of 5 to 30 cm. Specifically, the ADM may be designed to have slits on its surface at regular intervals of 1 to 20 mm to remove the possibility of generating a dead space, and to increase the area by 20 to 100%.

In one embodiment, the acellular skin substitute for breast reconstruction may include a circular base part; and
three or more protrusions that protrude outward from the circumference of the base part and are provided at different positions along the circumferential direction of the base part. Here, a plurality of slits may be formed radially along the circumferential direction of the base part based on the center of the base part in the base part and at least one of the protrusions, each slit may have a start point and an endpoint, and an angle between a first imaginary line extending from the center of the base part to the center of the slit and a second imaginary line connecting the start and end points of the slit may be 60 to 120°.

In the present invention, step (e) is a process of cutting the frozen ADM into the shape and size, which have been set by the CAD program, using a cutting device.

In one embodiment, the ADM may be frozen for 1 to 6 hours on a cooling plate maintained at−30 to 0° C., and cut into the shape and size, which have been set by a design program, using a CNC circular blade. The cutting direction may be a clockwise or counterclockwise direction from the outermost part to the center. Alternatively, a method of cutting from the center to the outermost part may be used.

In the present invention, a process of covering and wrapping a breast reconstruction implant with the acellular skin substitute prepared in the present invention, and then fixing it with surgical thread may be further performed.

In one embodiment, after wrapping a breast reconstruction implant with the acellular skin substitute, and fixing (suturing) protrusions with surgical thread, the resulting product may be inserted into the breast reconstruction site. Here, the suturing may be performed by a method of fixing the protrusions, and specifically, end parts of the ADM with surgical thread.

In one embodiment, an acellular skin substitute that can sufficiently wrap a 300 to 600 cc breast reconstruction implant is placed at the center of the implant and stretched to the extent that the slits in the dermis are widened, and pulled so that the protrusion ends (star-shaped corners) of the skin substitute come together on the back side of the implant. The ends of the pulled acellular skin substitute may be connected with surgical thread to prevent the possibility of the ADM detaching from the implant during the process of being inserted into a patient's breast and to help maintain the shape of the implant after insertion.

The present invention will be described in further detail regarding the following examples. However, the scope of the present invention is not limited to the following examples, and it will be understood by those of ordinary skill in the art that various modifications, alterations or applications are possible without departing from the technical details derived from the details described in the accompanying claims.

### EXAMPLES

### Example 1. Preparation of Acellular Skin Substitute (ADM)

### (1) Skin Pretreatment

Skin tissue (collected from a cadaver donated by a tissue bank for non-profit patient treatment) was prepared.

The cleaned skin tissue was placed on a clean bench, and using a sterilized scrapper, fascial tissue, fat tissue and other foreign substances attached to the dermis were removed until the dermis portion was seen. The remaining fat in the dermis was removed as much as possible using a sterilized scrapper, and the remaining unnecessary tissue was removed using sterilized scissors and tweezers. The resulting product was washed several times with sterile distilled water.

### (2) Removal of Epidermis and Cells

As a decellularization solution for removing the epidermis and cells, a 0.05 to 0.5M NaOH solution was used.

The fat-removed skin tissue was put into the NaOH solution and stirred at room temperature for 1 to 6 hours. After stirring, the skin tissue was taken out and the epidermis and debris were removed with a scrapper. The skin tissue from which the epidermis and cells were removed was washed 10 times with distilled water, and stirred and washed with distilled water at room temperature at 150 rpm for 1 to 6 hours (preparation of acellular skin substitute).

Experimental Example 1. Confirmation of the Presence or Absence of Cells and Tissue Damage after the Removal of Epidermis and Cells

The presence or absence of cells in the ADM prepared by the removal of the epidermis and cells ((2) of Example 1) was confirmed, and DNA was quantified.

First, to confirm the presence or absence of cells in the ADM, the epidermis and cell nuclei were stained with immunohistochemical staining and DAPI staining, respectively.

Specifically, to conduct the immunohistochemical staining, slides were manufactured by making the ADM into a paraffin block. After removing paraffin from the manufactured slides, antigen retrieval was conducted to sequentially attach primary and secondary antibodies, and then detected using an optical microscope.

In addition, to confirm the presence or absence of cells, cell nuclei were stained using 4′,6-diamidino-2-phenylindole (DAPI).

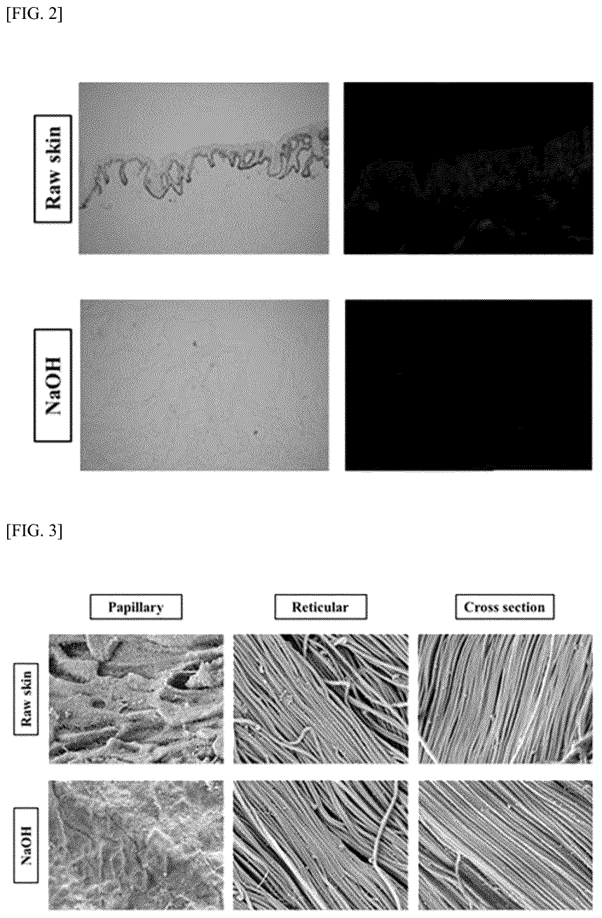

FIG. 2 shows the results obtained by observing the epidermis and cell nuclei after immunohistochemical staining and DAPI staining, respectively, using an optical microscope and a fluorescence microscope, to confirm the removal of the epidermis and cells from skin tissue after treatment with a decellularization solution.

In the Raw skin, the epidermis was stained brown, and cell nuclei were stained blue. As shown in FIG. 2, it can be confirmed that, in the Raw skin, a lot of blue DAPI is expressed. On the contrary, it can be confirmed that as cells are removed from the skin tissue (NaOH) treated with the decellularization solution, a part that is expressed in blue hardly appears. That is, it can be confirmed that the epidermis and cells shown in the Raw skin were removed after treatment with the decellularization solution.

Meanwhile, after treatment of the decellularization solution, the deformation or damage to collagen fibers is confirmed by photographing the ADM prepared by the removal of the epidermis and cells ((2) of Example 1) through SEM.

As shown in FIG. 3, it can be confirmed that, like the Raw skin, tissue deformation or damage is not observed even in skin tissue (NaOH) treated with a decellularization solution.

In addition, to quantify DNA, the ADM was chopped. The chopped dermis was treated with protease K, heated at 56° C. overnight to completely dissolve, and then treated with the same amount of phenol-chloroform. After centrifuging at 4° C., the supernatant was collected, mixed with sodium acetate with a 1/10 volume of the supernatant and 100% ethanol, and then stored at −20° C. for 30 minutes. Afterward, a DNA pellet was prepared using centrifugation, the supernatant was discarded, 70% ethanol was added and centrifugation was performed again to wash DNA pellet, thereby obtaining DNA with high purity. After naturally drying ethanol thoroughly, the resulting product was dissolved in distilled water, and then DNA was quantified using Nanodrop.

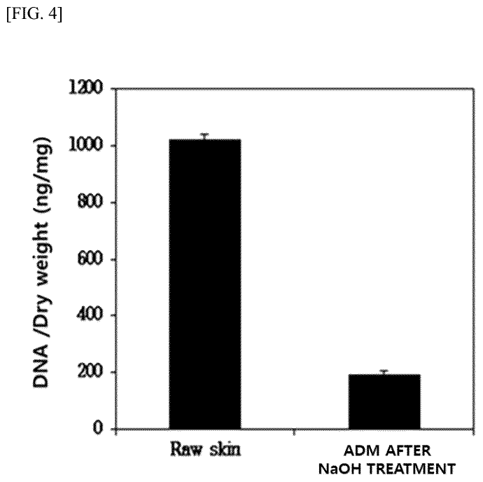

FIG. 4 shows the result of quantifying DNA extracted from the ADM.

As shown in FIG. 4, compared to the Raw skin, it can be confirmed that, in the ADM (ADM after NaOH treatment) prepared by treatment with a decellularization solution, the amount of DNA is significantly decreased. That is, after treatment with a decellularization solution, it can be confirmed that cells are removed from the skin.

### Example 2. Preparation of Acellular Skin Substitute that can Wrap and Fix Breast Reconstruction Implant

### (1) CAD Drawing Production Process

Through the illustration process, a design for wrapping a breast reconstruction implant was devised. First, when making the design, the operation time at a surgical site may be reduced to a minimum and efficiency is maximized by using a minimum acellular skin substitute. To this end, after setting the size of an acellular skin substitute that can wrap the breast reconstruction implant as a unit, an acellular skin substitute was designed to increase its area 20 to 100% by cutting slits and corners using a CNC machine.

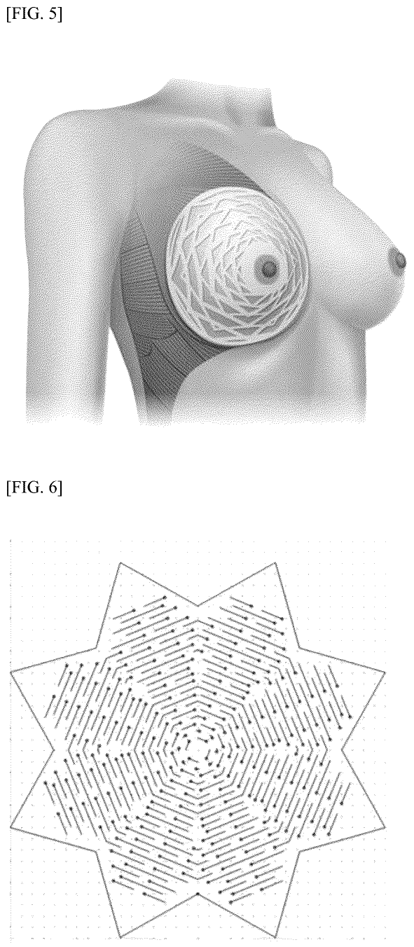

FIG. 5 shows the technical illustration of an acellular skin substitute having a design and shape that enables wrapping of a breast reconstruction implant.

When the acellular skin substitute has the design shown in FIG. 5, an operation time may be reduced to a minimum at a surgical site, and a minimum amount of acellular skin substitute can be used, so efficiency can be maximized.

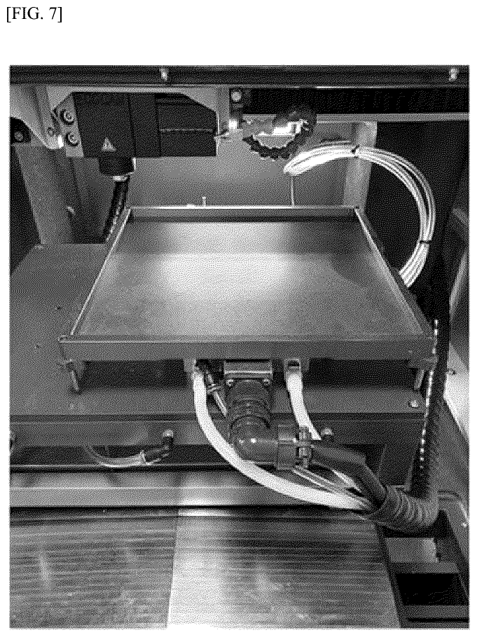

In addition, in this Example, based on FIG. 5, a CAD drawing expressing the shape of the acellular skin substitute for breast reconstruction that can wrap and suture the breast reconstruction implant was made (FIG. 6).

Considering that the volumes of domestic and foreign breast reconstruction implants are 300 to 600 cc, the acellular skin substitute output from a CNC machine was able to be manufactured to have a diameter of 5 to 25 cm. In addition, since the area can be increased due to slits, the size of the implant was not affected at all.

### (2) Process of Cutting Acellular Skin Substitute Using CNC Machine

The acellular skin substitute prepared in Example 1 is a biomaterial with high flexibility and tensile strength, and is soft and tough without being hard. For this reason, it is difficult for the blade of the CNC machine to cut the acellular skin substitute within an expected cutting range. To solve this problem, as shown in FIG. 7, a cooling plate (P200525-HS-100) that can be maintained at−5 to −25° C. was used.

The acellular skin substitute prepared in Example 1 was placed on the cooling plate, and water was added to reach 100% of the thickness of the acellular skin substitute and then frozen.

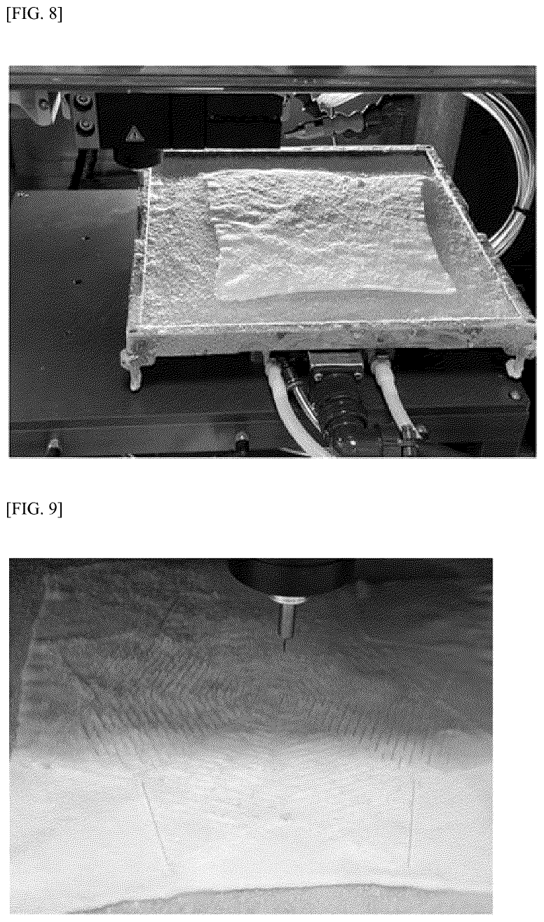

FIG. 8 shows a frozen acellular skin substitute, and when the acellular skin substitute is frozen, not only the blade of a CNC machine may cut the acellular skin substitute, but also the frozen water may fix the acellular skin substitute, so the acellular skin substitute may not shake and may be cut into a designed shape.

Subsequently, the acellular skin substitute was cut according to the CAD drawing prepared in (1) using a CNC machine (Hyeopsung Dynamics Co., Ltd., M4D-K) (FIG. 9).

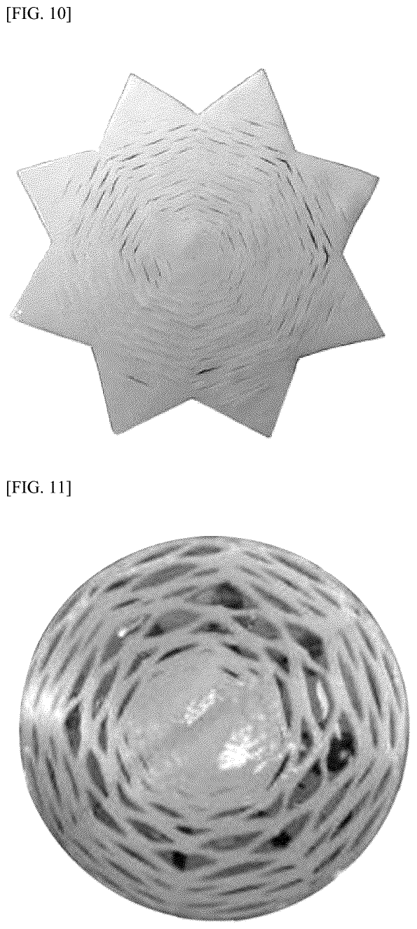

FIG. 10 shows an acellular skin substitute cut according to the CAD drawing.

The acellular skin substitute may be used for breast reconstruction.

### (3) Process of Wrapping and Fixing Breast Reconstruction Implant

A breast reconstruction implant has a size of 300 to 600 cc to fit a patient's original breast size. Likewise, to wrap various sizes of breast reconstruction implants, an acellular skin substitute was manufactured to have a diameter of 5 to 25 cm. In addition, on the surface of the acellular skin substitute, comb-patterned slits formed at regular intervals of 1 to 20 mm were made to be easily enlarged, and a dead space was not generated to reduce seroma side effects.

Specifically, the breast reconstruction implant was wrapped with the acellular skin substitute manufactured in (2). In the breast reconstruction implant, when the side where a nipple is located is called an upper side, and the side located on the side of a patient's ribs is called the back side, the upper surface was covered with the skin substitute, and then turned over to the back side to gather the ends of the protrusions of the acellular skin substitute.

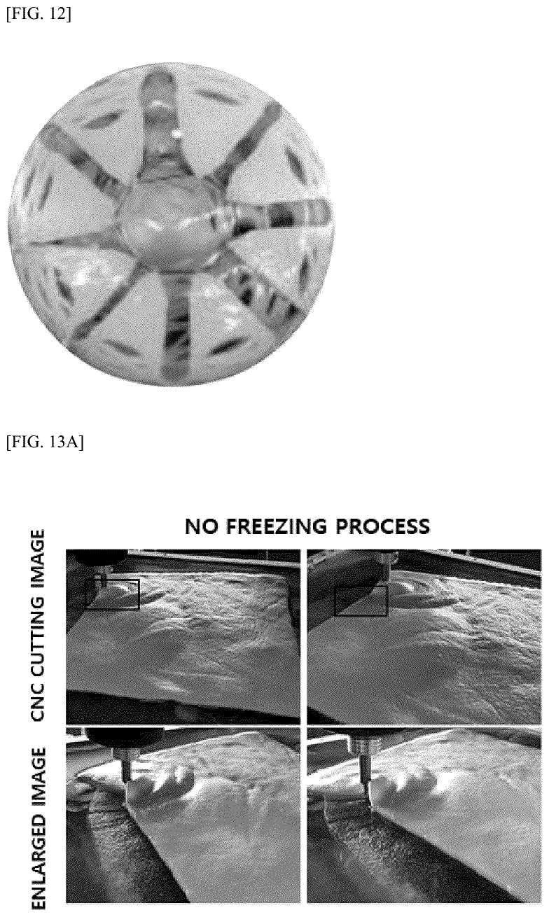

FIG. 11 shows that the upper surface of a breast reconstruction implant is covered with an acellular skin substitute, and FIG. 12 shows that ends of the protrusions of the acellular skin substitute are fixed with absorbent surgical thread, polyglyconate suture Maxon (Covidien), to cover the back side of the breast reconstruction implant.

### Experimental Example 2. Evaluation of Acellular Skin Substitute According to Freezing Process

In (2) of Example 2, CNC cutting performance was evaluated according to whether or not the freezing process was performed.

The acellular skin substitute subjected to the freezing process represents one that was placed on a cooling plate, and subjected to a freezing process for 1 to 6 hours after pouring water, as shown in (2) of Example 2, and an acellular skin substitute not subjected to the freezing process represents one on which the above-described freezing process was not performed.

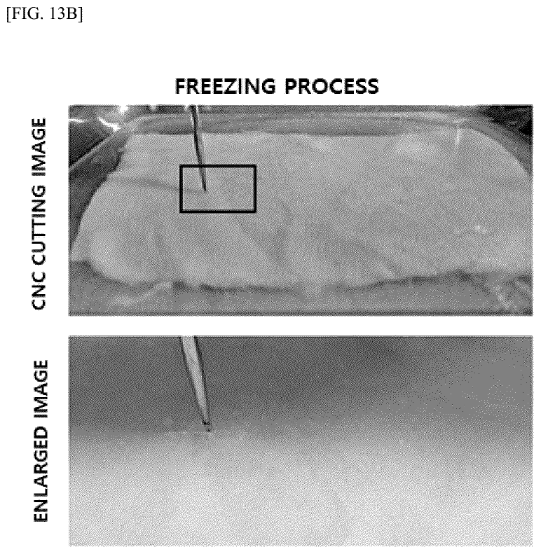

FIG. 13 is a set of photographs of cutting an acellular skin substitute subjected to a freezing process and an acellular skin substitute not subjected to the freezing process using a CNC machine.

As shown in FIG. 13, when a freezing process according to the present invention was not performed, since tissue was not fixed, the surrounding tissue was pulled and the tissue was pushed and not cut (FIG. 13A). However, when a freezing process was performed, it can be confirmed that, as water was frozen, tissue was fixed and therefore was able to be cut into a desired shape (FIG. 13B).

### INDUSTRIAL APPLICABILITY

In the present invention, an acellular skin substitute may be prepared through the removal of the epidermis and an acellularization process, in which the use of a proteolytic enzyme and a surfactant is excluded.

Since the acellular skin substitute is cut into suitable sizes and shapes to wrap a breast reconstruction implant through a design program and then cut with a CNC machine, the time for breast reconstruction surgery may be reduced, and since no dead space is formed after suturing, the incidence of seromas may be reduced. Accordingly, the side effects of surgery may be lessened.

1-22. (canceled)

23. A method of preparing an acellular skin substitute for breast reconstruction, comprising:
placing an acellular dermal matrix (ADM) on a cooling plate maintained at −30° C. to 0° C.;
adding water onto the cooling plate to reach 20 to 150% of the thickness of the ADM and freezing the ADM; and
cutting the frozen ADM using a cutting device.

24. The method of claim 23, wherein the acellular skin substitute, comprising:
a circular base part; and
three or more protrusions which protrude outward from the circumference of the base part, and are provided at different positions along the circumferential direction of the base part,
wherein a plurality of slits are formed radially along the circumferential direction of the base part based on the center of the base part in the base part and at least one of the protrusions,
each slit has a start point and an end point, and
an angle between a first imaginary line extending from the center of the base part to the center of the slit and a second imaginary line connecting the start and end points of the slit is 60° to 120°.

25. The method of claim 24, wherein the radius of the base part is 3 cm to 10 cm.

26. The method of claim 24, wherein the number of protrusions is 4 to 12.

27. The method of claim 24, wherein one protrusion forms a surface by connecting lines starting from two arbitrary points on the circumference of the base part, and
does not intersect with another protrusion except for the two arbitrary points.

28. The method of claim 27, wherein the area of the protrusion decreases as the protrusion extends in a protruding direction.

29. The method of claim 27, wherein the protrusion has a triangular shape where two straight lines originating from two arbitrary points on the circumference of the base part meet.

30. The method of claim 27, wherein the length between the center point of the base part and the end of the protrusion is 3 cm to 15 cm.

31. The method of claim 27, wherein two arbitrary points on the circumference of the base part forming one protrusion and the center point of the base part form an imaginary triangle, and
the first imaginary line extending from the center point of the base part to the center point of the slit and the second imaginary line connecting the start point and the end point of the slit are perpendicular to each other in the imaginary triangle.

32. The method of claim 24, wherein 100 to 400 slits are formed.

33. The method of claim 24, wherein the slits have one or more forms selected from the group consisting of a straight line, a curved line, and a V shape.

34. The method of claim 24, wherein the length of the slit is 1 mm to 20 mm.

35. The method of claim 24, which is used for breast reconstruction, and the breast reconstruction implant is wrapped with the acellular skin substitute, and then the protrusions are fixed with surgical thread.

36. The method of claim 23, further comprising:
preparing an ADM by removing the epidermis and cells from skin tissue.

37. The method of claim 36, wherein the removal of the epidermis and cells from skin tissue is performed using a decellularization solution, and
the decellularization solution comprises one or more selected from the group consisting of sodium hydroxide, potassium hydroxide, ammonium hydroxide, calcium carbonate, magnesium hydroxide, calcium hydroxide, and ammonia.

38. The method of claim 23, wherein the freezing of the ADM is performed for 1 to 6 hours.

39. The method of claim 23, wherein the cutting of the frozen ADM using a cutting device is a process of cutting the ADM into the shape and size set by a design program.

40. The method of claim 23, wherein the cutting device is a CNC machine having a circular blade with a diameter of 0.1 mm to 5 mm.

41. A method of preparing an acellular skin substitute for breast reconstruction, comprising:
preparing an acellular dermal matrix (ADM) by removing the epidermis and cells from skin tissue;
placing the ADM on a cooling plate maintained at −30° C. to 0° C.;
adding water onto the cooling plate to reach 20% to 150% of the thickness of the ADM and freezing the ADM;
setting the shape and size of an acellular skin substitute that is able to wrap a breast reconstruction implant using a design program; and
cutting the frozen ADM into the shape and size set by the design program using a cutting device,
wherein the shape of the ADM set by the design program comprises a circular base part; and
three or more protrusions which protrude outward from the circumference of the base part, and are provided at different positions along the circumferential direction of the base part,
a plurality of slits are formed radially along the circumferential direction of the base part based on the center of the base part in the base part and at least one of the protrusions,
each slit has a start point and an end point, and
an angle between a first imaginary line extending from the center of the base part to the center of the slit and a second imaginary line connecting the start and end points of the slit is 60° to 120°.

42. The method of claim 41, further comprising:
wrapping the breast reconstruction implant with the acellular skin substitute for breast reconstruction.
